(19) United States
(12) Patent Application Publication
(10) Pub. No.: US 2012/0282594 A1
(43) Pub. Date: Nov. 8, 2012

(54) METHOD OF ENHANCED DETECTION FOR NANOMATERIAL-BASED MOLECULAR SENSORS

(75) Inventors: Gugang CHEN, Columbus, OH (US); Avetik R. HARUTYUNYAN, Columbus, OH (US)

(73) Assignee: HONDA MOTOR CO., LTD., Tokyo (JP)

(21) Appl. No.: 13/466,741

(22) Filed: May 8, 2012

Related U.S. Application Data
(60) Provisional application No. 61/483,733, filed on May 8, 2011.
(60) Provisional application No. 61/502,326, filed on Jun. 28, 2011.

(51) Int. Cl. G01N 27/02; C12M 1/34; G01N 27/22; G01N 21/75; B82Y 15/00

(52) U.S. Cl. 435/5; 436/116; 435/34; 436/86; 436/113; 436/94; 422/98; 422/83; 435/287.1; 435/288.7; 436/117; 977/700; 977/902

(57) ABSTRACT

Sensors based on single-walled carbon nanotubes and graphene which demonstrate extreme sensitivity as reflected in their electrical conductivity to gaseous molecules, such as NO, NO₂ and NH₃, when exposed to in situ ultraviolet (UV) illumination during measurement of the analytes are disclosed. The sensors are capable of detection limits of NO down to almost 150 parts-per-quadrillion (“ppq”), detection limits of NO₂ to 2 parts-per-trillion (“ppt”), and detection limits of NH₃ of 33 ppt.

### RELATED APPLICATIONS

The present application claims benefit from earlier filed U.S. Provisional Applications No. 61/483,733 filed May 8, 2011, and No. 61/502,326, filed on Jun. 28, 2011, the entire disclosures of which are incorporated by reference for all purposes.

### BACKGROUND

1. Field of the Invention

Sensing ultra-low level of gas molecules is critical for environmental monitoring, space exploration, industrial process controlling, homeland security, agriculture and medical applications. The sensitivity of a solid-state sensor typically depends on the surface area of the material used for the sensor. Use of carbon nanotubes (“CNT”) and graphene provides new opportunities for ultrasensitive and ultrafast electronic sensors due to their high surface-to-volume ratio and extraordinary electronic transport properties associated with the unique electronic structures resulting from the CNT's nanoscale one-dimensional hollow geometry which makes the electrical response to the adsorption of molecules extremely sensitive, and graphene's high crystalline single-atom thick two-dimensional structure. However, both CNT and graphene sensors are often unintentionally contaminated due to sample preparation and device fabrication. Unfortunately, some of these contaminants are not easily removed by traditional methods such as thermal annealing, and thus greatly reduce sensor performance. In addition, inevitable background species may also interfere with the target gas, whose contributions become increasingly important at very low analyte concentrations.

There are several methods to incorporate carbon nanotubes into sensor architecture such as direct growth, drop deposition, printing and dielectrophoresis.

2. Discussion of the Related Art

It is well known that nitrogen oxides including NO and NO₂ play an important role in the chemistry of our atmosphere. In addition, NO is an important signaling molecule that acts in many living tissues to regulate a diverse range of physiological and cellular processes. Abnormal amount of NO synthesis has been implicated in a variety of diseases. Therefore, the capability to detect and monitor extremely low level of those gases is very important. As a result, there has been rapid progress towards low concentration detection of NO and NO₂ on carbon nanotube based sensors.

Because pristine single-walled carbon nanotubes (“SWNTs”) were speculated to be relatively limited in sensor performance, much effort has been focused on functionalized nanotubes. Tin oxide coated multi-walled carbon nanotubes could detect NO down to 2 parts-per-million (“ppm”). SWNTs wrapped with a 3,4-diaminophenyl-functionalized dextran exhibited selectivity for in vivo detection of NO, while polymer coated SWNTs had shown NO detection limit (“DL”) of 5 parts-per-billion (ppb) compared to 300 ppb for bare SWNTs.

For NO₂ detection, a field-effect transistor (FET) sensor based on an individual semiconducting-SWNT (“S-SWNT”) demonstrated remarkable sensitivity to 200 ppm of NO₂ and a DL of 44 ppb had been achieved using a SWNT-based chemiresistor on prefabricated interdigitated gold electrodes. Sub-ppb sensing of NO₂ on polyethileneimine coated SWNTs has also been reported, although the authors pointed out that the lowest reliably detected concentrations were several ppb with a response time of 1 to 2 minutes. NO₂ detection down to ppb level had also been reported on other nanomaterials such as TiO₂ and TiO₂-PEDOT. Several sensing mechanisms have been explored including resistance, thermopower, capacitance, gas ionization, permittivity, microwave resonance, and surface acoustic wave.

### SUMMARY

The present disclosure is directed to a method for molecular analysis by providing a nanomaterial-containing sensor component, radiating the nanomaterial-containing sensor component with a predetermined wavelength, then contacting the nanomaterial-containing sensor component with a molecular analyte while radiating continues, and measuring a change in an electrical or physical property of the nanomaterial-containing sensor component during the contacting step.

The present teachings further include a molecular sensor composed of a nanomaterial-containing sensor region, a radiation source for radiating the nanomaterial-containing sensor region with a predetermined wavelength, an electrical connection to the nanomaterial-containing sensor region to measure a change in electrical properties of the nanomaterial-containing sensor region when in contact with an analyte species. The nanomaterial-containing sensor region is radiated with the predetermined wavelength and undergoes a change in electrical properties while in contact with the analyte species.

Also taught by the present application is a device for sensing the presence of an analyte of interest made up of a SiO₂/Si substrate, a nanomaterial-containing region located on one side of the SiO₂/Si substrate with two electrical contacts to the nanomaterial-containing region. A radiation applying device is also part of this device which further includes a measuring apparatus connected to the two electrical contacts to measure a change in conductance of the nanomaterial-containing region upon contacting the analyte. The radiation applying device can apply radiation during exposure of the nanomaterial-containing region to the analyte.

With the presently taught method of in situ cleaning with ultraviolet (UV) light illumination, a two-terminal graphene sensor can have ultrahigh sensitivity to nitric oxide (NO) with detection limit as low as 158 parts-per-quadrillion (“ppq”), while detection limit as low as 590 ppq is achieved on SWNT. Comparable gas sensing results on species such as NO₂ and NH₃ further confirmed the ultrasensitivity achieved with graphene and SWNT under UV radiation.

The sensitivity of the presently taught SWNTs containing sensors can reach a detection limit down to 590 parts-per-quadrillion (“ppq”) for NO gas and 1.51 parts-per-trillion (“ppt”) for NO₂ gas at room temperature. The SWNT sensor shows reproducible response and recovery with a time scale of minutes. It appears that this ultrahigh sensitivity is owing to the surface cleanness of SWNTs. Furthermore, the presently taught individual S-SWNT FET device demonstrates that sensitivity can be tuned by applying an external gate bias.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1A is an SEM image, and FIGS. 1B and 1C are graphical representations of current versus time and voltage for a sensor according to the present disclosure, and FIG. 1D is Raman spectra of the SWNT films;

FIGS. 2A, 2B, 2C, 2D, and 2E are graphical representations of conductance change versus time, and FIG. 2F is current versus time for a sensor according to the present disclosure;

FIGS. 3A and 3B are graphical representations of conductance change versus time for a sensor according to the present disclosure;

FIG. 4A is an AFM image, and FIG. 4B is a graphical representation of current versus time and gate voltage for a sensor according to the present disclosure;

FIGS. 5A, 5B, and 5C are graphical representations of conductance change versus time for a sensor according to the present disclosure;

FIG. 6A is a Raman spectra, 6B is current versus voltage characterization in vacuum, and 6C is a graphical representation of conductance change versus time for a sensor according to the present disclosure;

FIGS. 7A, 7B, 7C, and 7D are graphical representations of conductance change versus time for a sensor according to the present disclosure;

FIGS. 8A and 8B are graphical representations of conductance change versus time for a sensor according to the present disclosure, and

FIGS. 9A, 9B, 9C and 9D are graphical representations of conductance change versus time for a sensor according to the present disclosure.

### DETAILED DESCRIPTION

In more detail, the presently disclosed method for molecular analysis includes providing a nanomaterial-containing sensor component, radiating this nanomaterial-containing sensor component with a predetermined wavelength, contacting the nanomaterial-containing sensor component with a molecular analyte while radiating also continues, and then measuring a change in an electrical or physical property of the nanomaterial-containing sensor component during this contacting step.

The electrical or physical property changed by the contacting with the analyte can be, for example and without limitation, the conductivity, dielectric constant, dielectric strength, permeability, permittivity, piezoelectric constant, Seebeck coefficient, thermopower, capacitance, wave impedance, wave absorption, emission, luminescence, luminance, thermal conductivity, mechanical and optical properties of the nanomaterial-containing sensor component. By applying, for example, a voltage from a voltage source to the nanomaterial-containing sensor component it is possible to monitor the change in the electrical or physical property caused in the nanomaterial-containing sensor component upon contact with the molecular analyte. One property of particular interest includes the change in conductance of the nanomaterial-containing sensor component.

The radiation utilized by this method can include Gamma ray radiation, X-ray radiation, UV radiation, visible radiation, infrared radiation, microwave radiation, radio radiation, heat, and any other electromagnetic field sufficient to modify the electrical or physical property of the nanomaterial-containing sensor component. In one preferred method, the radiation can be UV radiation. The method can be conducted in a vacuum, in air, in an inert gas, or in any controlled environment.

As seen in the various examples set forth herein, the method can be used to measure the concentration or detect the existence of molecular analytes such as, for example, without limitation, NO, NO₂, NH₃, chemical molecules, biological molecules, viruses, bacterial, protein, DNA, RNA and any other substances that interact with nanomaterials. Of particular interest are NO, NO₂ and NH₃.

Suitable examples of nanomaterial-containing sensor components include a SWNT thin film, a SWNT thin film FET structure, an individual S-SWNT FET structure, a S-SWNT thin film FET structure, an individual graphene structure, a monolayer graphene structure, a multiple layered graphene structure, a graphene thin film structure, and any other structures involving nanoscale materials.

The present disclosure also teaches a molecular sensor made up of a nanomaterial-containing sensor region, a radiation source for radiating the nanomaterial-containing sensor region with a predetermined wavelength, and an electrical connection to the nanomaterial-containing sensor region to measure a change in electrical properties of the nanomaterial-containing sensor region when in contact with an analyte species. A suitable nanomaterial-containing sensor region for the presently disclosed molecular sensor can be radiated with the predetermined wavelength and can undergo a change in electrical properties while in contact with the analyte species.

The present molecular sensor can also have a voltage applying device arranged to apply a voltage to the nanomaterial-containing sensor region. The predetermined wavelength can include, for example, Gamma ray radiation, X-ray radiation, UV radiation, visible radiation, infrared radiation, microwave radiation, radio radiation, heat, and any other electromagnetic field sufficient to modify the electrical or physical property of the nanomaterial-containing sensor component.

For this molecular sensor the nanomaterial-containing sensor region can be made from, for example and without limitation, a SWNT thin film, a SWNT thin film FET structure, an individual S-SWNT FET structure, a S-SWNT thin film FET structure, an individual graphene structure, a monolayer graphene structure, a multiple layered graphene structure, a graphene thin film structure, and any other structures involving nanoscale materials.

The electrical or physical property of the nanomaterial-containing sensor region that can be changed by contact with the analyte species can be, for example, the conductivity, dielectric constant, dielectric strength, permeability, permittivity, piezoelectric constant, Seebeck coefficient, thermopower, capacitance, wave impedance, wave absorption, emission, luminescence, luminance, thermal conductivity, mechanical and optical properties of the nanomaterial-containing sensor region. Other physical properties can also be affected by the contact with the analyte. One property of particular interest includes the change in conductance of the nanomaterial-containing sensor region.

Another device disclosed herein includes a device for sensing the presence of an analyte of interest containing a SiO₂/Si substrate with a nanomaterial-containing region on one side of the SiO₂/Si substrate. Two electrical contacts can be in contact with the nanomaterial-containing region, as well as a measuring apparatus connected to the two electrical contacts to measure a change in conductance of the nanomaterial-containing region upon contacting the analyte. Additionally, a radiation applying device that can provide a predetermined wavelength to the nanomaterial-containing region while it is in contact with the analyte can be a part of the presently disclosed device.

In this device, the nanomaterial-containing region can be made to include, for instance, a SWNT thin film, a SWNT thin film FET structure, an individual S-SWNT FET structure, a S-SWNT thin film FET structure, an individual graphene structure, a monolayer graphene structure, a multiple layered graphene structure, a graphene thin film structure, and any other structures involving nanoscale materials.

In one preferred device, the nanomaterial-containing region can be a graphene layer which can be synthesized by chemical vapor deposition on Cu foil. This exemplary device can have two electrical contacts made of layers of Au and Ti with respective thicknesses of 120 nm and 30 nm. These layers of Au and Ti can be applied sequentially to the graphene layer by vacuum thermal deposition. The device can then undergo an annealing treatment by heating at 350° C. for two hours under an Ar environment.

The presently disclosed device can use a UV light source as its radiation applying device. Furthermore, the device can be configured to allow the nanomaterial-containing region to be radiated with a predetermined wavelength while in contact with the analyte of interest.

According to the present teachings, in situ exposure with UV radiation can significantly improve gas sensitivity for both CNT and graphene. Application of in situ UV light illumination on the graphene surface during the course of detection a millimeter size graphene sensor can achieve a detection limit to toxic gaseous molecules as low as 158 ppq at room temperature.

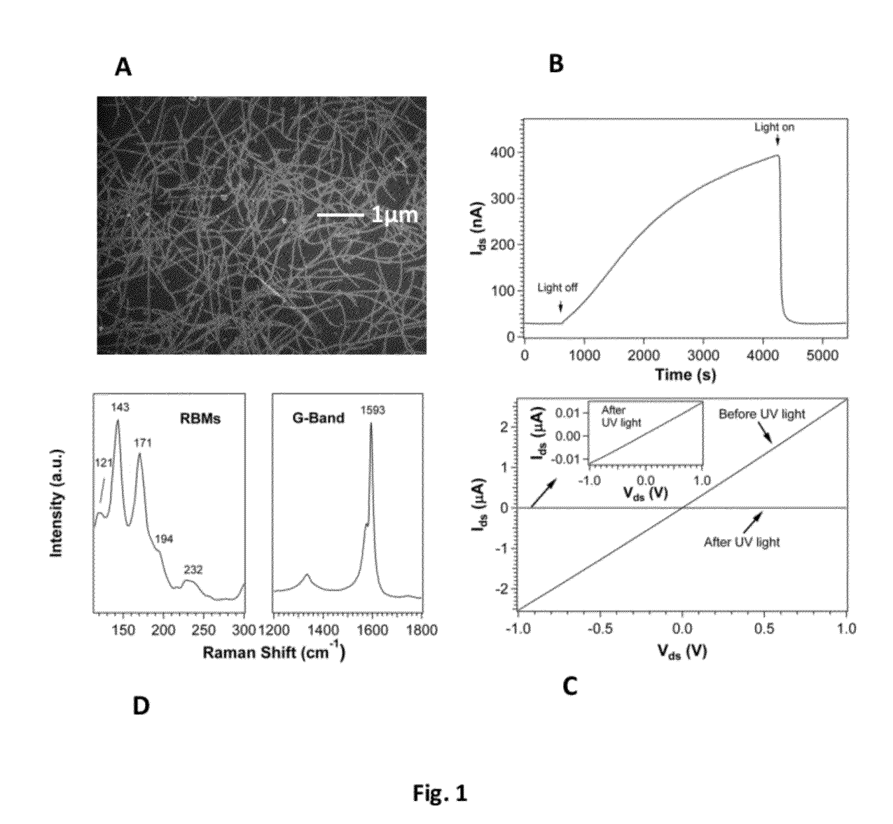

In the present disclosure, two types of sensors in FET geometry were fabricated. The first one was based on a SWNT thin film (FIG. 1A) and the second one was based on an individual S-SWNT. In both cases, the SWNTs were obtained from direct growth by chemical vapor deposition using iron nanocatalysts supported on SiO₂/Si substrate. Atomic force microscopy and Raman analysis indicate that the tubes are SWNTs with diameters in the range of 1-2 nm. FIG. 1D shows a typical Raman spectrum taken with 532 nm laser excitation and averaged from 50 micron-sized spots of the SWNT film.

FIG. 1A illustrates scanning electron microscopy image of the thin film, FIG. 1B is the typical response of I_{ds} current to UV light under flowing N₂, and FIG. 1C illustrates I-V characteristics of the presently disclosed SWNT device before and after UV light illumination. The data before UV light illumination was taken in air, while the data after UV light was recorded in an inert atmosphere under UV light exposure after thorough cleaning of SWNTs. The inset on FIG. 1C shows the same curve plotted on an enlarged scale.

A ten fold current change was observed owing to the UV illumination (FIG. 1B) after the sample had been heat treated under vacuum and then exposed to pure N₂ in a sealed gas flow system. In contrast, the current change is about 200 fold when comparing the current flowing through the sensor under UV light in an inert atmosphere to that when the sensor was exposed in air (FIG. 1C).

Present theory, without being limited thereto, provides the apparent explanation that the SWNTs are so sensitive to their environment that any minor imperfection in the sample sealing system or low level of interactive impurities in the N₂ gas will be enough to disturb the nanotubes intrinsic conductance drastically. These results demonstrate high sensitivity of cleaned SWNTs and the crucial role of surface cleanness prior to chemical sensing. In order to take advantage of this intrinsic sensitivity, in the present application the SWNTs had been continuously illuminated with UV light throughout the course of gas detection.

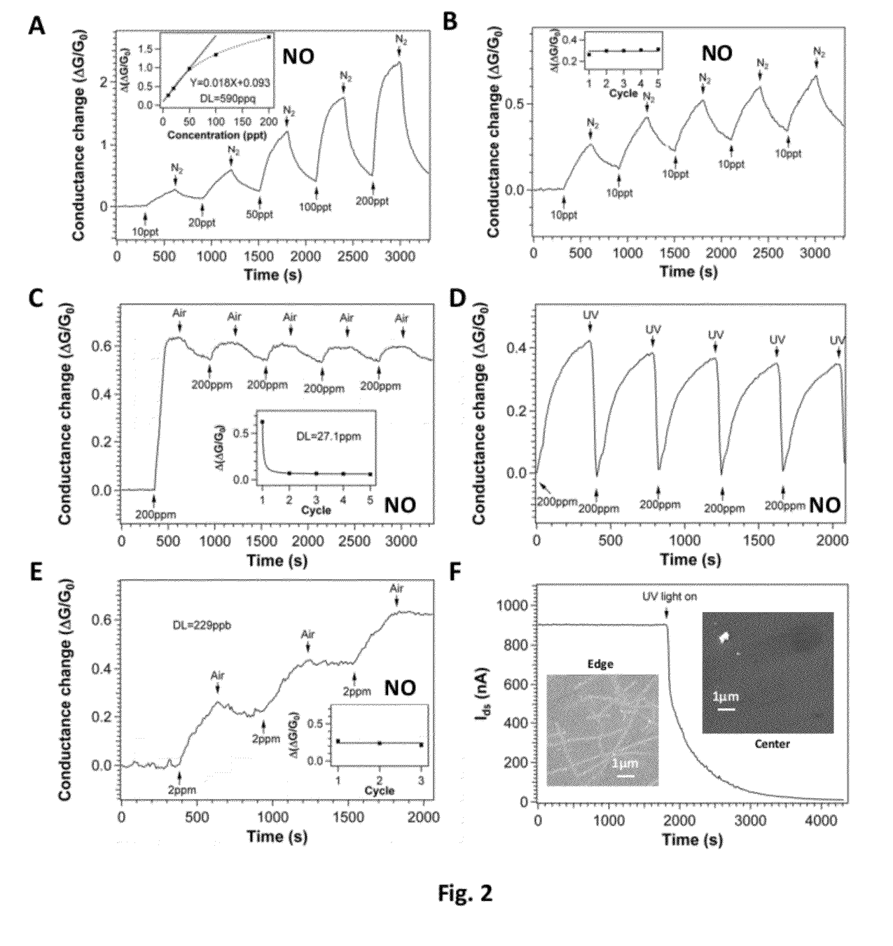

The electrical response of a SWNT film sensor under in situ UV light illumination according to the present teachings to NO molecules is shown in FIG. 2A. An unambiguous response is observed at a 10 ppt concentration of NO. A linear response had been observed with concentrations up to 50 ppt (FIG. 2A inset). Further increase of NO level leads to non-linear behavior, presumably indicating that the NO coverage had passed beyond a weak perturbation regime where a different phase of gas adsorption had occurred. The DL can be derived from the root-mean-square (rms) noise of the baseline and the slope of ΔG/G₀ versus concentration. To calculate the rms noise, a third-order polynomial equation was used to fit ΔG/G₀ versus time. A noise value of 0.00354 was thus determined by comparing the experimental data with the fitted curve. DL=3 rms/slope with a signal-to-noise ratio (s/n) of 3 used for the DL estimation. A slope or sensitivity of 0.018 was derived from a linear fit segment from the graph in FIG. 2A, that gives DL=590 ppq. The sensor response was studied by cycling between pure N₂ and 10 ppt NO (FIG. 2B). FIG. 2B shows the conductance change versus time recorded with the SWNT film cycling with 10 ppt of NO. A very reproducible result with a signal of 0.29±0.03 had been observed.

The effect of UV light illumination on the performance of a presently taught SWNT sensor in dry air is displayed in FIG. 2C. The relative change of conductance (ΔG/G₀) versus time with repeated 200 ppm of NO exposures is shown in FIG. 2D. The inset shows the amplitude of signal at each cycle. DL is estimated to be 27.1 ppm. The sensor's response was recorded as the change of conductance normalized by the initial conductance commonly defined as the sensitivity. After the electrical conductance of the sample had been stabilized, the device was exposed to 200 ppm of NO. Without UV light, the sensor had a very big (about 0.6 or 60%) and fast response (about 10 seconds) to the first NO exposure, while subsequent exposures had much smaller (about 7%) but quite reproducible response. This smaller response at later cycles was attributed to partial device recovery which appears to have many active sites still occupied by pre-adsorbed NO molecules. FIG. 2D shows that the application of UV light illumination during air flushing not only dramatically reduced the recovery time down to a few seconds but also enhanced the signal 5 times, that is, from about 7% to about 36%. NO sensing with continuous in situ UV light illumination shows much improved sensitivity, where estimated DL improves about 2 orders of magnitude from 27.1 ppm down to 229 ppb (FIG. 2E). The life time of the sensor is currently believed to depend on the thickness of the SWNT film, that is, thicker SWNT films appeared to last longer. Electrical study shows that the SWNT sensor continuously loses conductance under UV light illumination. SEM images taken after about 1 hour of illumination show the center of the film with few SWNTs left while the edge of the sample has much lowered density of SWNTs than before the UV light was applied (FIG. 2F). The SEM images taken from the edge and center regions of the film are shown in the lower left and upper right panels, respectively.

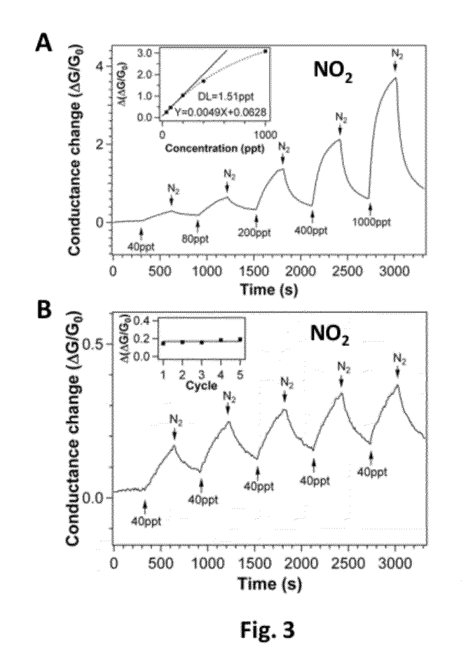

The presently disclosed sensors can also be used to measure NO₂ molecules in a nitrogen flow (FIG. 3). Similar to NO detection, a linear response had been observed at low NO₂ concentrations. The conductance was observed to increase 25% with 40 ppt of NO₂ exposure in 5 minutes, and the device was 80% recovered in 2 minutes. The inset shows sensor response with concentration. The DL at s/n=3 was estimated to be 1.51 ppt. Like the detection of NO molecules the sensing and recovery cycling experiments at 40 ppt NO₂ showed similar reproducibility (FIG. 3B).

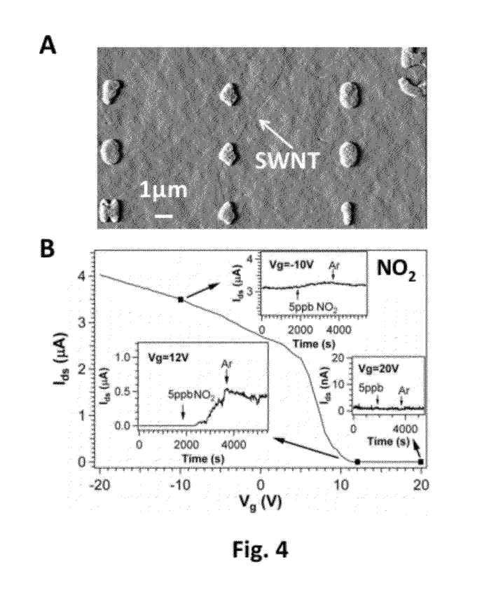

The sensitivity dependence of a SWNT-FET device according to the present teachings to ambient species with an applied electrical gate voltage is also presented herein. Importantly this feature can open the prospect for selective detection of various analytes. An individual S-SWNT based device was prepared (FIG. 4), with a heavily doped Si substrate used as the back gate. The utilized S-SWNT appeared to be p-type before molecular sensing (FIG. 4B). The FET device was exposed to 5 ppb of NO₂ at three different gate voltages, −10 V, 12 V and 20 V, FIG. 4B insets, respectively).

a) V_{g}=−10 V, where the I_{ds} versus V_{g} curve has a small slope value (|dI_{ds}/dV_{g}|<0.1 μS). As a result, the sensor showed very weak response to 5 ppb of NO₂.

(b) V_{g}=12 V, where the V_{g} was right before the threshold of a sharp slope (|dI_{ds}/dV_{g}|>0.4 μS). The exposure to 5 ppb of NO₂ increased the current about 300 fold in several minutes.

(c) V_{g}=20V, where |dI_{ds}/dV_{g}|˜0. Correspondingly, the sensor showed no obvious response at this gate bias.

As presently disclosed, these results can be understood taking into account that, as an electron acceptor, NO₂ is known to be a strong oxidizer due to an unpaired electron and thereby the adsorption of NO₂ is equivalent to applying a negative chemical gate voltage. At V_{g}=20V, the device was at an “off” state where a corresponding chemical gate induced by 5 ppb of NO₂ exposure was not enough to shift the device to a more conducting state. At V_{g}=−10V, the device showed weak response since the NO₂ adsorption shifted the I_{ds}=F(V_{g}) curve more away from the large derivative of |dI_{ds}/dV_{g}|. The gate voltage Vg=12V allowed to exploit the sharp change of I_{ds} vs V_{g} upon NO₂ adsorption and thus the highest sensitivity was observed. Therefore, by setting up the initial conducting state by application of an electrical gate bias, it is disclosed herein that one can tune and maximize the sensitivity of a SWNT based sensor. Moreover, according to this analysis, the sensor is supposed to respond differently to molecules with opposite electron affinities. This effect can be carefully utilized for selective detection. Hence, the results in FIG. 4B demonstrate that it would be possible to tune the sensitivity and introduce selectivity, at least to some degree, by proper adjusting the gate bias of a SWNT-FET sensor.

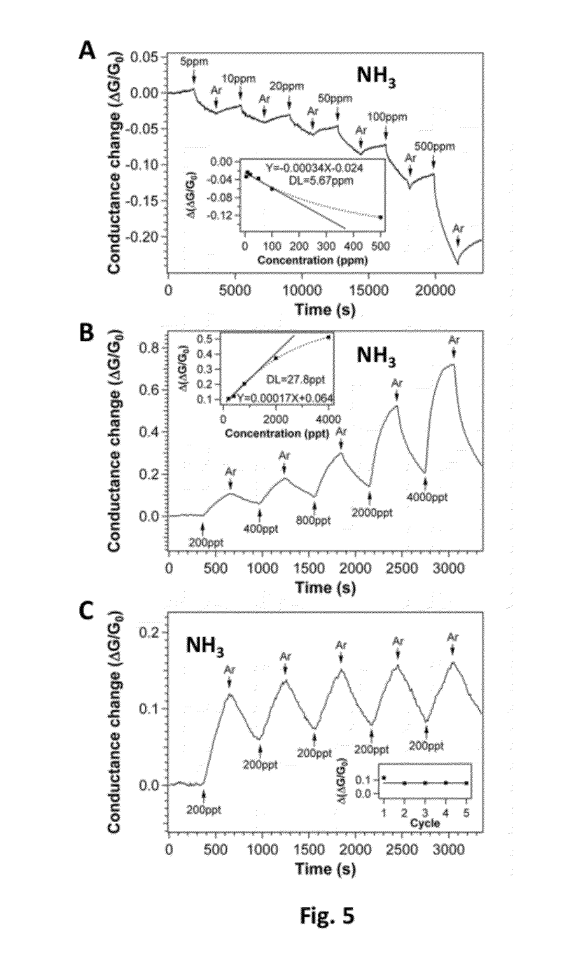

The presently disclosed method can also be utilized to detect NH₃. Interestingly, the change of conductance to NH₃ exposures reverses its direction, that is, from a decrease of conductance to an eventual increase of conductance upon application of in situ UV light illumination on the sensor (FIG. 5). This change of sign is attributed to the initial conducting state of the sensor. Carbon nanotubes have been found to be p-type in air. In contrast to NO and NO₂ which are electron acceptors, NH₃ is an electron donor. Because of a lone electron pair that can be donated, negative charge transfer from NH₃ adsorption will cancel the existing p-type carriers of an air doped nanotube and thus make its conductance decrease. After applying UV light illumination, FIG. 5B indicates that the UV light has efficiently removed the p-type dopants so as to shift the Fermi level of a nanotube closer to the carrier neutrality point. Therefore, any charge transfer from subsequent NH₃ adsorption will only increase its conductance by adding free electrons. Through the presently disclosed method of applying in situ UV light illumination the DL of NH₃ improves under otherwise identical sensing conditions from 5.67 ppm to 27.8 ppt, nearly 5 orders of magnitude sensitivity improvement in this case.

Specifically, FIG. 5A illustrates the conductance change versus time recorded with the sensor exposed to increasing NH₃ concentrations without UV light illumination. The inset shows sensor response with concentration. DL is estimated to be 5.67 ppm. Results from the presently disclosed method are illustrated in FIG. 5B, where the sensor response under in situ UV light illumination provides a DL estimated to be 27.8 ppt. FIG. 5C shows the reproducibility of the sensor response at 200 ppt of NH₃ exposures under in situ UV light illumination.

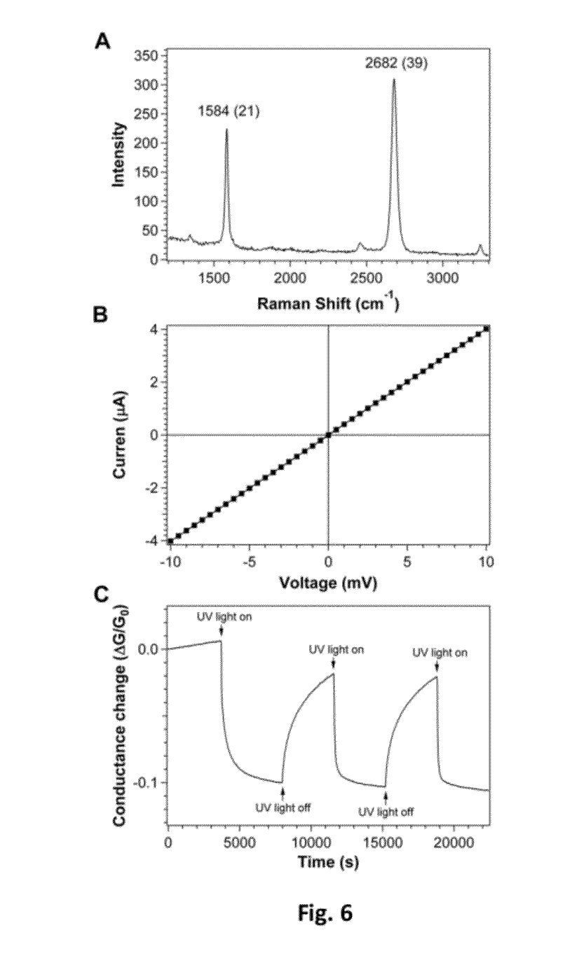

The graphene used in the presently disclosed method can be synthesized by chemical vapor deposition. FIG. 6A shows a typical Raman spectrum averaged from 50 different spots of the graphene with 532 nm laser excitation (laser spot size ˜1 μm on the sample). The data was taken in vacuum with minimum laser power in order to avoid thermal heating of the sensor.

The presence of weak D (about 1330 cm⁻¹) and D′ bands (about 1620 cm⁻¹) indicates a high quality of the graphene. The prominent intensity and sharpness of the 2D peak suggest that the presently taught sensor is likely composed of a single-layer graphene. Typical current versus voltage characteristics of the device is shown in FIG. 6B. The straight line I-V relationship indicates a good low omega contact between graphene and its two metal contacts. No obvious hysteresis had been observed on the device independent of presence of gas analytes.

As presently disclosed the effect of UV light illumination on the response of graphene under flowing N₂ is shown in FIG. 6C. The sensor response (about 10%) is very reproducible with UV light on and off. Similar response had been observed when the graphene was in vacuum (about 10⁻³ Torr). Since the decrease of conductivity with UV exposure clearly excludes the possibility of an UV light induced photoelectrical effect, the present understanding without being limited thereto, is that because graphene is so sensitive to its environment minute levels of impurities can interact and cause the increase in conductance. Possible sources of impurities include minor imperfections in the sample sealing system or low levels of interactive impurities in the inert carrier gas which can dope the graphene and increase its conductance after the UV exposure is stopped. Fortunately, the effect from those unintentional contaminants can be reversed by the presently taught method of utilizing in situ UV light illumination (see FIG. 6C).

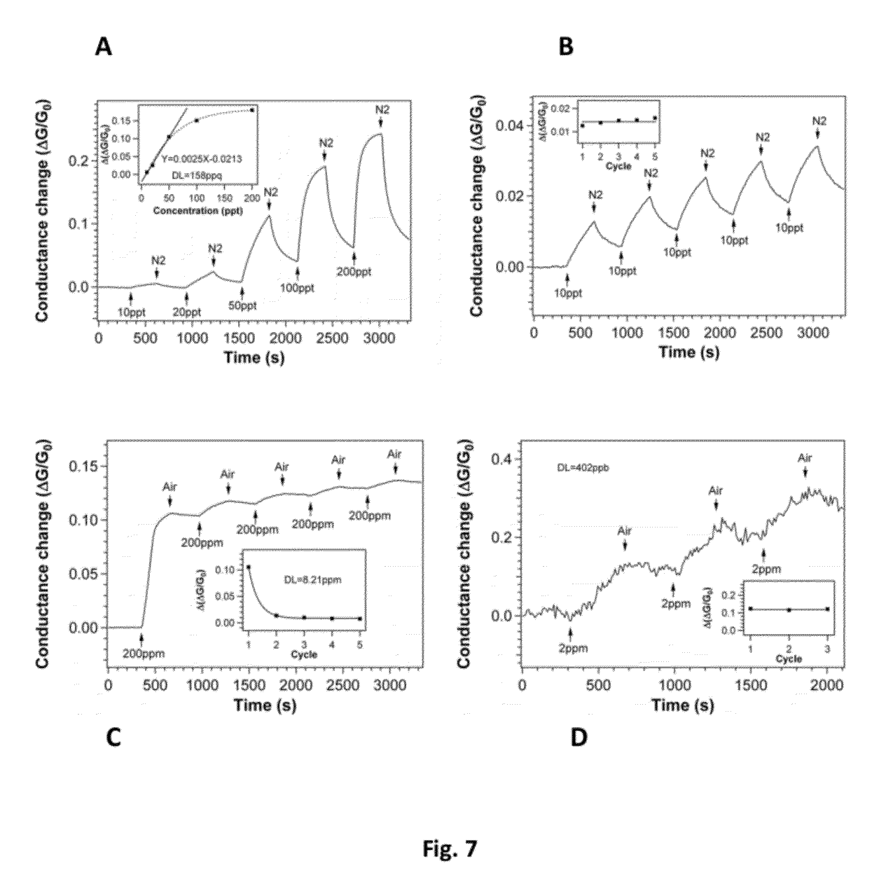

The present method is further illustrated in FIG. 7 which shows the electrical response of graphene under in situ UV light illumination to NO molecules. A linear response was observed with NO concentrations up to 50 ppt (FIG. 7A inset). Further increase of NO level led to a non-linear behavior, presumably indicating that a different phase of gas adsorption had occurred. The detection limit is estimated to be 158 ppq at a signal-to-noise ratio (s/n) of 3 from the root-mean-square (rms) noise of the baseline (ΔG/G₀ versus time) and the slope of ΔG/G₀ versus concentration using DL=3 rms/slope, where the rms noise is 1.32×10⁻⁴ and the slope is 0.0025 in this case.

FIG. 7A shows the relative change of conductance (ΔG/G₀) versus time recorded with NO exposures ranging from 10 to 200 ppt. The inset shows sensor response with NO concentration.

The reproducibility of the presently taught method on sensor response while cycling between pure N₂ and 10 ppt NO is illustrated in FIG. 7B. Very repeatable results with a s/n about 100 were observed at 10 ppt of NO exposure. The inset shows the amplitude of response at each cycle.

FIGS. 7C and 7D illustrate NO detection in air, unlike the above flowing N₂ results, without and with in situ UV illumination, respectively. Using the presently taught method of in situ UV illumination, provides an increase in DL from 8.21 ppm to 402 ppb.

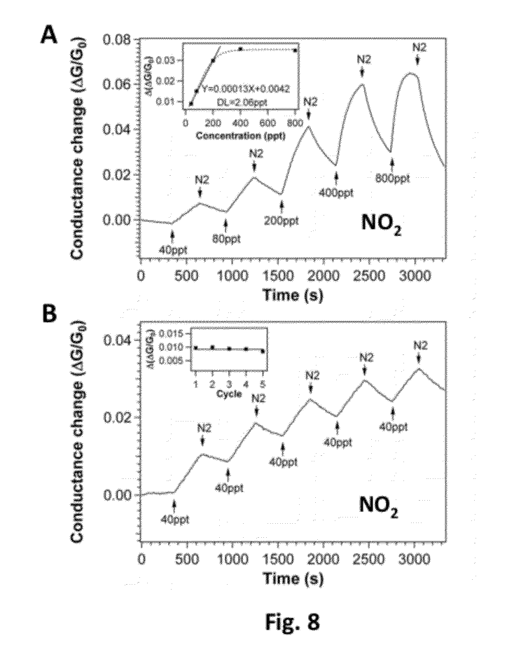

Similar performance had been observed for NO₂ molecules in a nitrogen flow with in situ UV radiation. (FIG. 8). The conductance increased ˜1% with 40 ppt of NO₂ exposure in 5 min. The DLs at s/n=3 were estimated to be 2.06 ppt. As seen in the detection of NO molecules above, the sensing and recovery experiments at 40 ppt of NO₂ exposure show excellent reversibility and reproducibility.

FIGS. 8A and 8B illustrate the results of sensing under in situ UV light illumination, as presently disclosed herein. FIG. 8A plots the conductance change versus time recorded with NO₂ exposures ranging from 40 to 1000 ppt. The inset shows sensor response with NO₂ concentration. FIG. 8 B provides the reproducibility of sensor response at 40 ppt of NO₂. The inset shows the sensor response at each cycle.

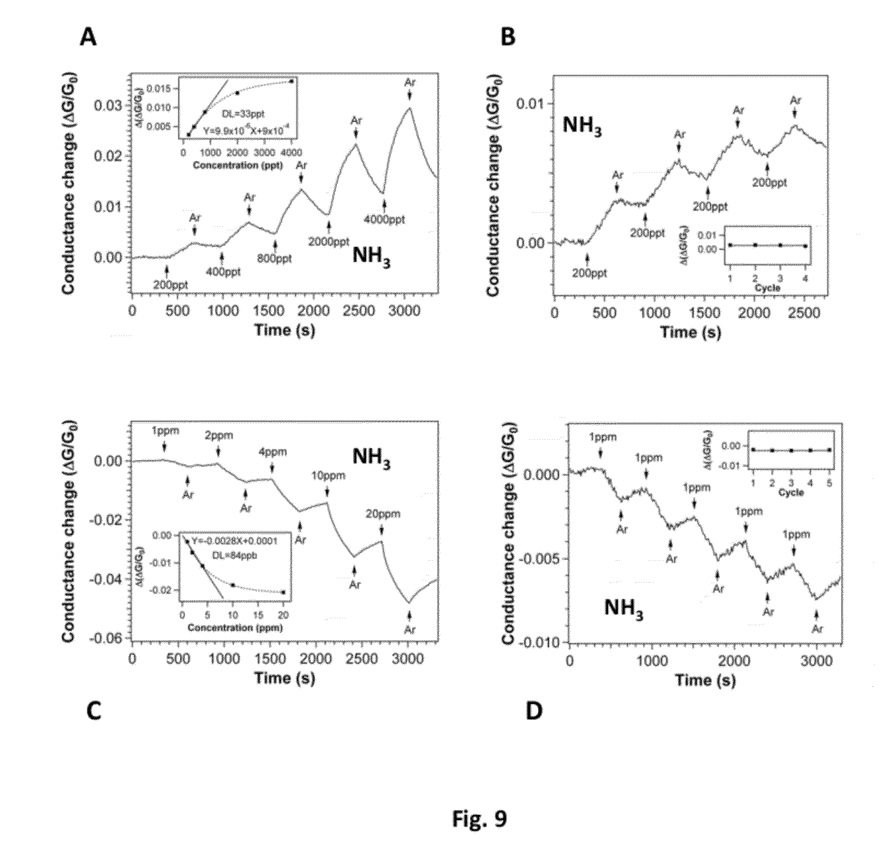

The conductance response of graphene to NH₃ molecules under the presently disclosed method is the opposite of that observed for both NO and NO₂, that is, initially a decrease in conductance to a subsequent increase in conduction when under in situ UV radiation, see FIG. 9.

Specifically, FIG. 9 illustrate the sensor responses (conductance change (ΔG/G₀) versus time) to NH₃ with and without in situ UV light illumination. FIG. 9A provides the sensor response under in situ UV light illumination. The inset shows sensor response with concentration. The detection limit is estimated at 33 ppt. FIG. 9B shows the sensor reproducibility at 200 ppt of NH₃ exposure under in situ UV light illumination. The inset shows the change of conductance at each cycle. Similar sensing experiments without UV light were shown in FIGS. 9C, and 9D, where the detection limit is estimated to be 84 ppb.

While conductivity is proportional to the product of carrier density and mobility, the relative contribution of these two factors is unclear without further experimental and theoretical investigations. The present theory, without being limited thereto, is that this change of sign is related to the initial conducting state of graphene. Graphene and carbon nanotubes were found to be p-type conductors in air mainly due to the presence of oxygen. Because NH₃ has a lone electron pair that can be donated, negative charge transfer from NH₃ adsorption will cancel the existing positive carriers (holes) of graphene and thus make its conductance decrease. Contrasting FIGS. 9A and 9B (with in situ UV radiation) with FIGS. 9C and 9D (without in situ UV radiation) indicate that UV light illumination has efficiently removed the p-type dopants so as to shift the Fermi level of graphene close to the neutrality point. Any charge transfer from subsequent NH₃ adsorption will only increase its conductance by adding free electrons. The present disclosure shows that upon exposing the graphene device to UV radiation, the detection limit of NH₃ improves from 84 ppb to 33 ppt; an unexpected 3 orders of magnitude enhancement.

### Experimental Details

The graphene (˜1×3 mm²) was synthesized by chemical vapor deposition on Cu foil, followed by Cu etching and graphene transferring onto SiO₂/Si substrate. Two simple electrical contacts (Au/Ti with thicknesses of 120 nm/30 nm) were applied on top of graphene through vacuum thermal deposition. The device was then annealed at 350° C. for two hours under an Ar environment.

The sensor response was monitored by the current flowing through the CNT sensor or graphene sheet at 25° C. (referred to as “room temperature”) as the sensor was exposed to a gas analyte. Research-grade (99.9999% purity) nitrogen (N₂) or argon (Ar) was used as the carrier gas. A total flow rate of 1000 ml/min was used for all experiments. Gas dilution was carried out by a simple two-flow mixing of carrier and analyte gases through carefully calibrated digital mass flow controllers (Brooks Model #5850S). The flow accuracy of these MFCs is ±0.7% of flow rate and ±0.2% of full scale. Gas dilution was carried out from certified 50 (200) ppb mixture gas of NO (NO₂) in N₂, where the concentrations were confirmed by chemiluminescence measurements at an accuracy of ±5% (Matheson Tri-Gas Inc.). For NH₃, certified 1±0.1 ppm NH₃ in Ar was used as the starting gas before dilution, whose concentration was determined by gravimetric method (The American Gas Group). Both MFCs were calibrated using a multipoint calibration curve with gas measurement equipment (DHI) accurate to 0.01 ml/min. Based on these specifications and the gas flow rates used, the error is estimated to be ±21% at the lowest analyte gas level, while the accuracy improves at higher concentrations.

In situ UV light illumination via a pencil-shape UV light (λ=253.7 nm, and I˜1.7 mW/cm²) was applied through a quartz window on a customized flowing cell that has electrical feedthrough connections for temperature and electrical measurements. Gas sensing experiments were carried out at a 10 mV driving voltage where the current was monitored with a Keithley 4200-SCS instrument as the sensor was exposed to a gas analyte. All detection experiments were done at a fixed room temperature of 25° C. through a temperature controller equipped with automatic heating and cooling assemblies.

Additional discussion on the experimental techniques, results and theory related to performance of the presently taught methods and devices can be found in Chen, G., Paronyan, T. M., Pigos, E. M., and Harutyunyan, A. R.; “Enhanced gas sensing in pristine carbon nanotubes under continuous ultraviolet light illumination,” Sci. Rep. 2, 343; DOI:10.1038/srep00343 (2012), the complete disclosure incorporated by reference herein in its entirety for all purposes.

All publications, articles, papers, patents, patent publications, and other references cited herein are hereby incorporated by reference herein in their entireties for all purposes.

Although the foregoing description is directed to the preferred embodiments of the present teachings, it is noted that other variations and modifications will be apparent to those skilled in the art, and which may be made without departing from the spirit or scope of the present teachings.

The foregoing detailed description of the various embodiments of the present teachings has been provided for the purposes of illustration and description. It is not intended to be exhaustive or to limit the present teachings to the precise embodiments disclosed. Many modifications and variations will be apparent to practitioners skilled in this art. The embodiments were chosen and described in order to best explain the principles of the present teachings and their practical application, thereby enabling others skilled in the art to understand the present teachings for various embodiments and with various modifications as are suited to the particular use contemplated. It is intended that the scope of the present teachings be defined by the following claims and their equivalents.

What we claim is:

1. A method for molecular analysis comprising:
providing a nanomaterial-containing sensor component;
radiating the nanomaterial-containing sensor component with a predetermined wavelength;
contacting the nanomaterial-containing sensor component with a molecular analyte while radiating continues, and
measuring a change in an electrical or physical property of the nanomaterial-containing sensor component during the contacting step.

2. The method according to claim 1, wherein the electrical or physical property of the nanomaterial-containing sensor component comprises at least one member selected from the group consisting of conductivity, dielectric constant, dielectric strength, permeability, permittivity, piezoelectric constant, Seebeck coefficient, thermopower, capacitance, wave impedance, wave absorption, emission, luminescence, luminance, thermal conductivity, mechanical properties and optical properties.

3. The method according to claim 1, further comprising
applying a voltage from a voltage source to the nanomaterial-containing sensor component to thereby monitor the change in the electrical or physical property caused in the nanomaterial-containing sensor component upon contact with the molecular analyte.

4. The method according to claim 1, wherein radiating comprises exposure to radiation selected from the group consisting of Gamma ray radiation, X-ray radiation, UV radiation, visible radiation, infrared radiation, microwave radiation, radio radiation, heat, and any other electromagnetic field sufficient to modify the electrical or physical property of the nanomaterial-containing sensor component.

5. The method according to claim 4, wherein the radiation comprises UV radiation.

6. The method according to claim 1, wherein the molecular analyte comprises at least one member selected from the group consisting of NO, NO₂, NH₃, chemical molecules, biological molecules, viruses, bacterial, protein, DNA, RNA and any other substances that interact with nanomaterials.

7. The method according to claim 1, wherein the method is conducted in a vacuum, in air, in an inert gas, or in any controlled environment.

8. The method according to claim 1, wherein the nanomaterial-containing sensor component comprises at least one member selected from the group consisting of a SWNT thin film, a SWNT thin film FET structure, an individual S-SWNT FET structure, a S-SWNT thin film FET structure, an individual graphene structure, a monolayer graphene structure, a multiple layered graphene structure, a graphene thin film structure, and any other structures involving nanoscale materials.

9. A molecular sensor comprising:
a nanomaterial-containing sensor region;
a radiation source for radiating the nanomaterial-containing sensor region with a predetermined wavelength, and
an electrical connection to the nanomaterial-containing sensor region to measure a change in electrical or physical properties of the nanomaterial-containing sensor region when in contact with an analyte species,
wherein the nanomaterial-containing sensor region is radiated with the predetermined wavelength and undergoes a change in electrical or physical properties while in contact with the analyte species.

10. The molecular sensor according to claim 9, further comprising
a voltage applying device arranged to apply a voltage to the nanomaterial-containing sensor region.

11. The molecular sensor according to claim 9, wherein the predetermined wavelength comprises radiation selected from the group consisting of Gamma ray radiation, X-ray radiation, UV radiation, visible radiation, infrared radiation, microwave radiation, radio radiation, heat, and any other electromagnetic field sufficient to modify the electrical or physical property of the nanomaterial-containing sensor region.

12. The molecular sensor according to claim 9, wherein the nanomaterial-containing sensor region comprises at least one member selected from the group consisting of a SWNT thin film, a SWNT thin film FET structure, an individual S-SWNT FET structure, a S-SWNT thin film FET structure, an individual graphene structure, a monolayer graphene structure, a multiple layered graphene structure, a graphene thin film structure, and any other structures involving nanoscale materials.

13. The molecular sensor according to claim 9, wherein the electrical or physical property of the nanomaterial-containing sensor region comprises at least one member selected from the group consisting of conductivity, dielectric constant, dielectric strength, permeability, permittivity, piezoelectric constant, Seebeck coefficient, thermopower, capacitance, wave impedance, wave absorption, emission, luminescence, luminance, thermal conductivity, mechanical properties and optical properties.

14. A device for sensing the presence of an analyte of interest comprising
a SiO₂/Si substrate;
a nanomaterial-containing region on one side of the SiO₂/Si substrate;
two electrical contacts to the nanomaterial-containing region;
a radiation applying device, and
a measuring apparatus connected to the two electrical contacts to measure a change in conductance of the nanomaterial-containing region upon contacting the analyte.

15. The device according to claim 14, wherein the nanomaterial-containing region comprises at least one member selected from the group consisting of a SWNT thin film, a SWNT thin film FET structure, an individual S-SWNT FET structure, a S-SWNT thin film FET structure, an individual graphene structure, a monolayer graphene structure, a multiple layered graphene structure, a graphene thin film structure, and any other structures involving nanoscale materials.

16. The device according to claim 15, wherein the nanomaterial-containing region comprises a graphene layer.

17. The device according to claim 15, wherein the graphene layer comprises a graphene layer synthesized by chemical vapor deposition on Cu foil.

18. The device according to claim 16, wherein the two electrical contacts comprises layers of Au and Ti with respective thicknesses of 120 nm and 30 nm.

19. The device according to claim 18, wherein the layers of Au and Ti are applied sequentially to the graphene layer by vacuum thermal deposition.

20. The device according to claim 16, wherein the device is annealed at 350° C. for two hours under an Ar environment.

21. The device according to claim 14, wherein the radiation applying device comprises a UV light source.

22. The device according to claim 14, wherein the device is configured to allow the nanomaterial-containing region to be radiated with a predetermined wavelength while in contact with the analyte of interest.
